(19) United States
(12) Patent Application Publication
(10) Pub. No.: US 2005/0015132 A1
(43) Pub. Date: Jan. 20, 2005

(54) Combined transesophageal echocardiography and transesophageal cardioversion probe

(76) Inventor: Itzhak Kronzon, New York, NY (US)

Correspondence Address: FAY KAPLUN & MARCIN, LLP, 15O BROADWAY, SUITE 702, NEW YORK, NY, 10038, US

(21) Appl. No.: 10/826,199

(22) Filed: Apr. 15, 2004

Related U.S. Application Data
(60) Provisional application No. 60/463,834, filed on Apr. 16, 2003.

(51) Int. Cl. A61N 1/05

(52) U.S. Cl. 607/124

(57) ABSTRACT

A medical apparatus comprises a flexible probe for accessing a patient's esophagus via the mouth, the probe, when in an operative position extending from a proximal end which remains outside the patient to a distal end within the esophagus, an echocardiography transducer coupled to the distal end of the probe so that, when the probe is in the operative position, the echocardiography transducer is at a predetermined location within the esophagus relative to the heart to perform a transesophageal echocardiography procedure and an electrode disposed on the probe for delivering a cardioversion current to the heart via the esophagus.

### PRIORITY CLAIM

This Application claims the benefit of the U.S. Provisional Application Ser. No. 60/463,834 filed Apr. 16, 2003 which is expressly incorporated herein, by reference.

### BACKGROUND INFORMATION

Cardioversion is the standard of care for converting atrial fibrillation or atrial flutter to normal sinus rhythm. The standard method for administering cardioversion treatment is transthoracic, i.e., by application of cardioversion paddles to the exterior of the patient's chest to discharge electrical energy through the chest cavity of the patient to the heart. However, this is an inefficient procedure because most of the electrical energy discharged by the cardioversion device does not pass through the heart but is dissipated in extracardiac tissue, e.g., the chest wall and lungs.

Since most of the energy dissipates without reaching the heart, a higher amount of energy must be applied to the patient to assure that the required cardioversion energy passes through the heart. However, the higher energy may cause damage tissue, degrading patient safety and increasing discomfort.

### SUMMARY OF THE INVENTION

The present invention is directed to a medical apparatus comprising a flexible probe for accessing a patient's esophagus via the mouth. The probe, when in an operative position, extending from a proximal end which remains outside the patient to a distal end within the esophagus. The medical apparatus further includes an echocardiography transducer coupled to the distal end of the probe so that, when the probe is in the operative position, the echocardiography transducer is at a predetermined location within the esophagus relative to the heart to perform a transesophageal echocardiography procedure. The medical apparatus also includes an electrode disposed on the probe for delivering a cardioversion current to the heart via the esophagus.

Furthermore, a cardioversion mechanism comprising an electrode assembly selectively mountable to a transesophageal echocardiography probe, wherein, when mounted to the echocardiography probe, electrodes of the electrode assembly are fixed at a predetermined location with respect to the echocardiography probe, the electrode assembly being coupled to a power source for supplying a cardioversion current to heart via tissue located adjacent thereto when the echocardiography probe is in an operative position within an esophagus of a patient.

In addition, a method of treating a heart of a patient, comprising the steps of inserting into the patient's esophagus a device comprising a flexible probe having an echocardiography transducer coupled to a distal end thereof and at least one cardioversion electrode coupled to the probe, performing an echocardiography to analyze a condition of the heart and applying electric current to the at least one electrode to supply a cardioversion current to the heart when the echocardiography does not contraindicate cardioversion.

### BRIEF DESCRIPTION OF DRAWINGS

FIG. 1 shows the relationship between the esophagus and the heart of a normal patient;

FIG. 2 shows an exemplary transesophageal echocardiography device;

FIG. 3a shows an exemplary first production stage of an exemplary embodiment of a combined transesophageal echocardiography/transesophageal cardioversion (“TCC”) probe according to the present invention;

FIG. 3b shows an exemplary second production stage of an exemplary embodiment of a combined TCC probe according to the present invention;

FIG. 3c shows an exemplary final production stage of an exemplary embodiment of a combined TCC probe according to the present invention;

FIG. 4a shows an end view along a longitudinal axis of an exemplary electrode which may be used for the TCC probe according to the present invention;

FIG. 4b shows a side view of an exemplary electrode which may be used for the TCC probe according to the present invention;

FIG. 5a shows a first exemplary production stage of an exemplary wire lead for the TCC probe according to the present invention;

FIG. 5b shows a second exemplary production stage of an exemplary wire lead for the TCC probe according to the present invention;

FIG. 6a shows an exemplary transesophageal echocardiography device;

FIG. 6b shows an exemplary condom including transesophageal cardioversion elements according to the present invention;

FIG. 6c shows an exemplary TCC probe including the condom of FIG. 6b coupled to the transesophageal echocardiography device of FIG. 6a.

### DETAILED DESCRIPTION

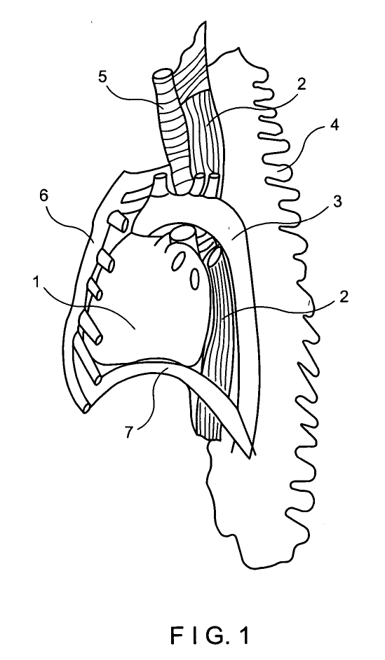

The present invention comprises a combined transesophageal echocardiography and transesophageal cardioversion probe. The exemplary embodiments of the present invention allow for the performance of both a transesophageal echocardiography and transesophageal cardioversion procedure using a single device that is inserted into the patient's esophagus. FIG. 1 shows the heart 1 contained within the pericardium, the sternum 6, the diaphragm 7, the trachea 5, the aorta 3, the esophagus 2 and the spinal column 4. As can be seen from FIG. 1, there is a close anatomical relationship between the esophagus 2 and the heart 1. This relationship is particularly close in the area of the left atrium. Therefore, it is possible to exploit this relationship by performing a cardioversion procedure from inside the esophagus, i.e., a transesophageal cardioversion.

By delivering cardioversion current via the esophagus, the electrical energy travels through less intervening tissue before reaching the heart. Thus application of a desired cardioversion current to the heart via transesophageal cardioversion is significantly more efficient than transthoracic cardioversion. In addition, a transesophageal cardioversion device may be more finely placed in relation to the heart in order to deliver electrical stimulation to specific areas of the heart. Thus, in comparison with transthoracic cardioversion, transesophageal cardioversion makes possible the achievement of superior results while passing a significantly lower amount of electrical energy through the tissues of the patient. Thus, both safety and patient comfort are improved. For example, transesophageal cardioversion may obtain results equivalent to transthoracic cardioversion while delivering as little as 10% of the energy (e.g., 30 Joules via transesophageal cardioversion versus 300 Joules needed for transthoracic cardioversion.

Transesophageal echocardiography is frequently performed prior to cardioversion procedures to determine whether conditions exist which indicate an increased risk to the patient if cardioversion were to be performed. For example, one such condition is the presence of blood clots in the heart which may indicate an increased risk of embolization following cardioversion.

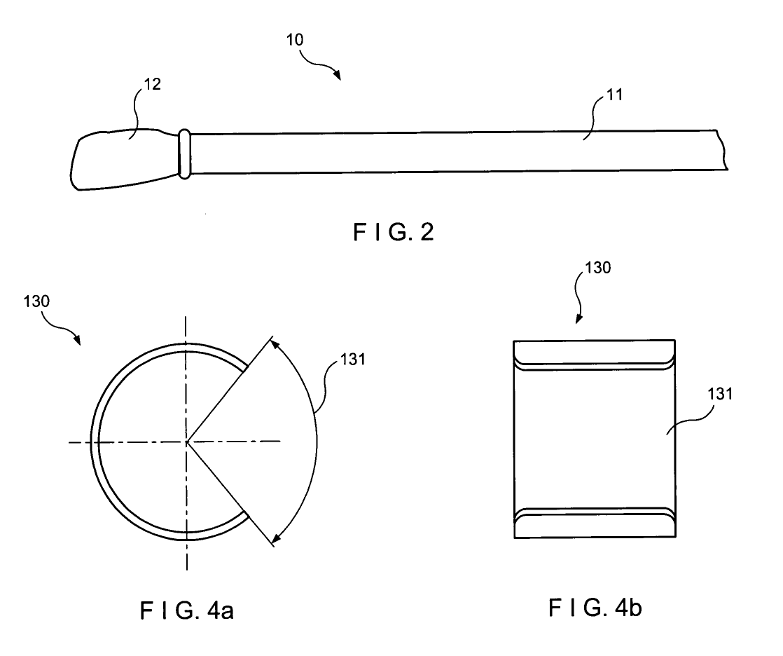

FIG. 2 shows an exemplary transesophageal echocardiography device 10 including a scope portion 11 and an echocardiography transducer 12. Typically, the patient is anesthetized and the transesophageal echocardiography device 10 is inserted into the patient's esophagus and maneuvered into a position such that the echocardiography transducer 12 is adjacent to the patient's heart. Echocardiography is then performed on the patient using the transesophageal echocardiography device 10. In known methods, the device 10 is then removed from the patient and the results are analyzed to determine whether any risk factors have been detected which contraindicate cardioversion. If no such factors are present, the patient anesthetized once again and a separate cardioversion procedure is performed with the same risks of sedation and, if the cardioversion is transesophageal, the risk and discomfort associated with the insertion of an endoscope into the patient's esophagus along with any required intubation, etc.

Thus, carrying out these procedures separately compounds the risks and discomfort to the patient. In addition, an increased risk may be generated if a blood clot forms after the completion of the cardioversion procedure because of the stunning of the atrium and the stagnation of the blood following the procedure. When the cardioversion and echocardiography are performed as two separate procedures, the status of the heart after the cardioversion is not known because there is no immediate echocardiographic monitoring performed. The exemplary embodiments of the present invention alleviate the need for two separate procedures because the transesophageal echocardiography device and the transesophageal cardioversion device are combined into a single probe which may perform both functions during a single insertion into the patient's esophagus.

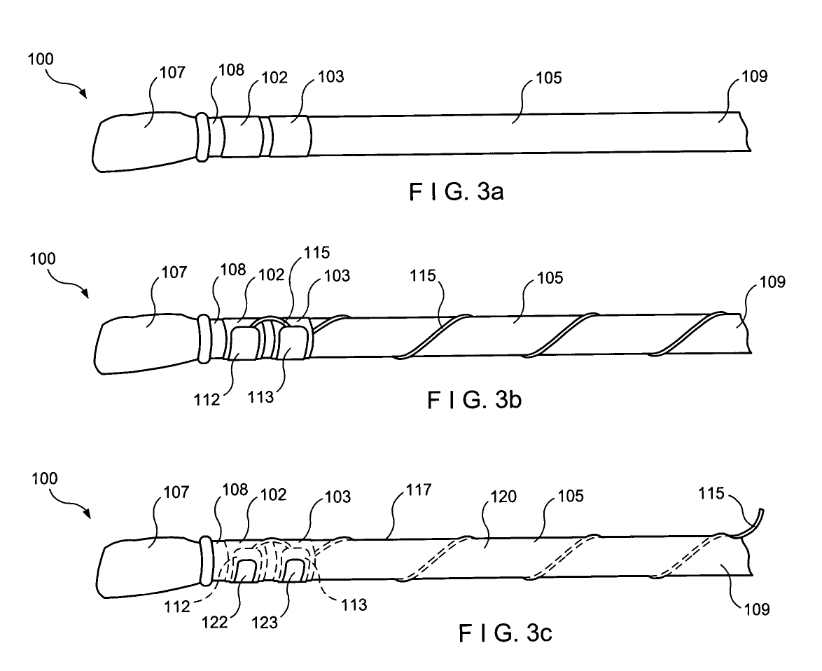

FIGS. 3a-c show a first exemplary embodiment of a combined transesophageal echocardiography/transesophageal cardioversion (“TCC”) probe 100 in various production stages. FIG. 3a shows a first stage of production of the TCC probe 100 including a scope portion 105 having a distal end 108 and a proximal end 109. An echocardiography transducer 107 is attached to the distal end 108 of the scope portion 105 resulting in a device similar to the transesophageal echocardiography device 10 described with reference to FIG. 2. In addition two tubes 102 and 103 have been placed around the scope portion 105 in the vicinity of the distal end 108. As will be described in greater detail below, these tubes 102 and 103 are placed in order to protect the scope portion 105 and the equipment contained in the scope portion 105 from the electrical energy generated by the transesophageal cardioversion portion of the TCC probe 100. The tubes 102 and 103 may be, for example, heat shrink tubing which is pre-stretched to fit over the echocardiography transducer 107 and then heated to tightly fit over the exterior of the scope portion 105.

Each of tubes 102 and 103 is approximately 10-13 millimeters (mm) in length and, in general, should be large enough such that the electrodes (described later) are completely contained within the bounds of the tubes 102 and 103. The first tube 102 is placed such that its distal end is approximately 4-6 mm proximal of the point where the echocardiography transducer 107 is attached to the distal end 108 of the scope portion 105. The purpose of placing the tubes 102 and 103 and the electrodes which will be placed on the tubes 102 and 103 in the vicinity of the echocardiography transducer 107 is to allow the transesophageal cardioversion portion of the TCC probe 100 to be accurately placed to provide electrical stimulation to desired areas of the heart, while maintaining the echocardiography transducer 107 in a location from which the echocardiography function may be simultaneously performed. The distal end of the second tube 103 may preferably be placed approximately 3 mm proximal of the proximal end of the first tube 102.

FIG. 3b shows the exemplary TCC probe 100 in a later production stage than FIG. 3a. FIG. 3b shows the same elements as FIG. 3a for the exemplary TCC probe 100 (i.e., scope portion 105, echocardiography transducer 107 and tubes 102 and 103), but in addition shows the placement of electrodes 112 and 113 and a wire lead 115. The wire lead 115 extends from the electrodes 102 and 103 at the distal end 108 of the scope portion 105, wrapping around the scope portion 105 to extend beyond the proximal end 109 of the scope portion 105 which remains outside the patient's body during use of the TCC probe 100. Thus, the wire lead 115 may be separated from the proximal end 109 of the scope portion 105 outside the body to couple to a power source for providing the electrical energy to perform the transesophageal cardioversion procedure. As would be understood by those of skill in the art, the power source may be any standard defibrillator. A more complete description of the wire lead 115 and its construction will be provided below.

As would be understood by those of skill in the art, the electrodes 112 and 113 maybe constructed from any conducting metal suitable for use within the body (e.g., titanium) to transmit the electrical energy to the tissue of the esophagus. As shown in FIG. 3b, the electrodes 112 and 113 are preferably placed in locations overlapping the tubes 102 and 103, respectively, such that when the electrodes are transmitting electrical energy no damage is caused to the scope portion 105 of the TCC probe 100. The tubes 102 and 103 are non-conducting and therefore electrically and physically isolate the scope portion 105 preventing the electrical energy from the electrodes 112 and 113 from burning the sheath of the scope portion 105. In this exemplary embodiment, each of the electrodes 112 and 113 is approximately 7-10 mm in length and fits within the confines of the corresponding one of the tubes 102 and 103. As a result of their size and the placement of the tubes 102 and 103, the distal end of the first electrode 112 is approximately 5-8 mm from the point where the echocardiography transducer 107 is coupled to the distal end 108 of the scope portion 105 and is more preferably between 5.5 and 8 mm from the point where the echocardiography transducer 107 is coupled to the distal end 108 of the scope portion 105. In this embodiment, the second electrode 113 is placed so that its distal end is spaced approximately 5-8 mm from the proximal end of the first electrode 112. As described above, the placement of the electrodes 112 and 113 is such that the TCC probe 100 may simultaneously perform the transesophageal echocardiography function and the transesophageal cardioversion function.

When the TCC probe 100 is performing the transesophageal cardioversion function, electrical energy will be transmitted by both of the electrodes 112 and 113 as shown by the fact that a portion of the wire lead 115 electrically connects the electrodes 112 and 113 to one another. The electrodes 112 and 113 may completely encircle the tubes 102 and 103 or they may be constructed partially cylindrically so that they can be clipped into place over the tubes 102 and 103. As will be described in greater detail below, the wire lead 115 is constructed with exposed conductors and crimp tubes to which the electrodes may be welded to form both a strong electrical connection to transmit the electrical energy and a strong physical connection so that the electrodes 112 and 113 will remain in place when the TCC probe 100 is used on a patient. The electrodes 112 and 113 are connected to the wire lead 115 prior to connecting the electrodes 112 and 113 to the scope portion 105. The electrodes 112 and 113 may then be connected to the scope portion 105 and adjusted to the correct spacing as required. Those skilled in the art will understand that the operator may apply cardioversion current to specific portions of the heart by further inserting the TCC probe 100 into, or partially withdrawing the TCC probe 100 from the esophagus until the electrodes 112 and 113 are in a desired position relative to the targeted portion of the heart.

FIGS. 4a-b show two views of an exemplary electrode 130 which may be used for the TCC probe 100. The exemplary electrode 130 (which may be used for either or both of the electrodes 112 and 113 as shown in FIG. 3b) is a type of electrode which may be snapped over the crimp tube of the wire lead 115. The end view along the longitudinal axis as shown in FIG. 4a shows that electrode 130 is C-shaped with an opening 131 of approximately 110 degrees allowing the electrode to be snapped into place. In this exemplary embodiment, the electrode 130 has an inner diameter of approximately 11 mm and a wall thickness of approximately 0.25-0.40 mm. Of course, those skilled in the art will understand that, depending on the dimensions of the underlying echocardiography unit, these dimensions may be varied in any manner required to allow the electrode to be bonded thereto. The side view as shown in FIG. 4b shows the details of the electrode 130 in the area of opening 131. As shown in this view, the electrode 130 has rounded comers to prevent damage to the esophagus.

In the exemplary embodiment shown in FIGS. 3b-c, two electrodes 112 and 113 are used to apply the energy for the transesophageal cardioversion procedure to the esophageal tissue. Those of skill in the art will understand that any number of additional electrodes may be included in TCC probe 100 according to this invention coupled with any number of independent wire leads to allow the electrodes to be individually energized or energized in selected groups. Alternatively, the TCC probe 100 may include only a single electrode. Additional electrodes may be coupled to the TCC probe 100 in the same manner described for attaching the electrodes 112 and 113. These additional electrodes may also be separated by distances similar to those described above in regard to electrodes 112 and 113. Maximizing the surface area of the electrodes maximizes the energy transfer during the transesophageal cardioversion procedure. However, those skilled in the art will understand that adding electrodes to a length that exceeds the length of the heart may result in an increase of the distribution of energy to extracardiac tissue.

In addition, as more electrodes are added, or the size of the electrodes is increased, the ability to move the electrodes within the esophagus to apply energy to a selected area of the heart may be diminished. A solution to this problem, and an alternative exemplary embodiment of the present invention, is to apply a series of electrodes along the length of the scope portion 105. The doctor may then selectively turn electrodes on and off to target energy to specific areas of the heart.

FIG. 3c shows the final production phase of the TCC probe 100. FIG. 3c shows the same elements as described above for FIG. 3a-b for the exemplary TCC probe 100 (i.e., scope portion 105, echocardiography transducer 107, tubes 102 and 103, electrodes 112 and 113 and wire lead 115), but in addition shows the placement of a wrapping tube 120. The wrapping tube 120 is used to cover all the additional material added to incorporate the transesophageal cardioversion portion into the TCC probe 100 so that irritation to the esophagus is minimized. The wrapping tube 120 may, for example, comprise silicone rubber tubing having a hardness of 50 shore or any other suitable coating.

As shown in FIG. 3c, the wrapping tube 120 extends from the proximal end 109 of the scope portion 105 which will remain outside the esophagus up to the distal end 108 where the echocardiography transducer 107 is attached. The wire lead 115 is shown covered by the wrapping tube 120 up to the proximal end 109 of the scope portion 105 where the wire lead 115 separates from the endoscopic portion in order to allow connection to the power source. In the area of the electrodes 112 and 113, two windows 122 and 123 are cut into the wrapping tube 120 to expose the electrodes 112 and 113 to transmit the cardioversion current to the esophageal tissue. As would be understood by those skilled in the art, the wrapping tube 120 may be adhered to the scope portion 105 at the point of attachment for the echocardiography transducer 107 and the windows 122 and 123 using medical adhesive.

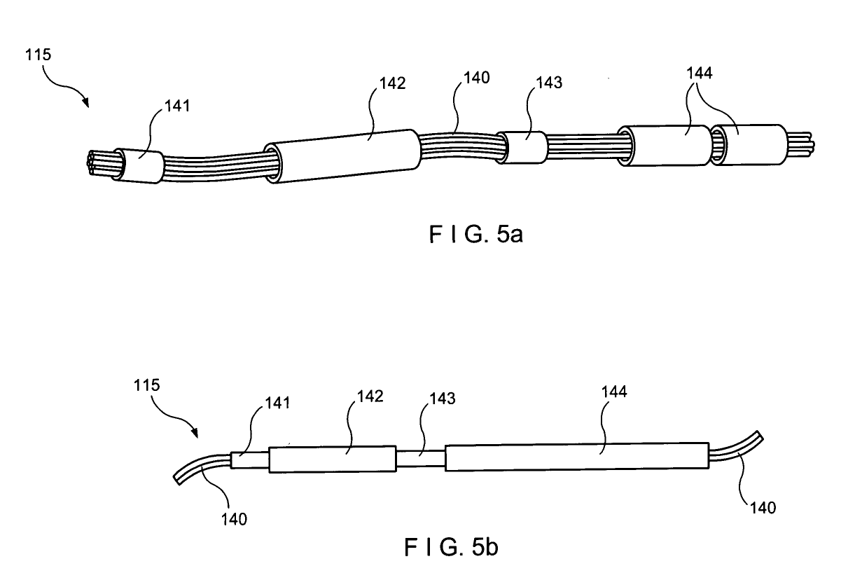

FIGS. 5a-b show an exemplary wire lead 115 for the TCC probe 100 in two stages of production. The wire lead 115 may contain multiple cables 140 (e.g., 16 cables) extending the entire length of the wire lead 115. The cables 140 may be, for example, copper conductors having a diameter of 0.001 inches surrounded by an ETFE coating. The wire lead 115 may be constructed by inserting the cables 140 through a first crimping ring 141. The crimping ring 141 may be, for example, a platinum-iridium ring having a length of 2-4 mm, a wall thickness of 0.1 mm and an inside diameter of 1.0-1.4 mm.

After the crimping ring, a first piece of tubing 142 is placed over the cables 140. The length of the first piece of tubing 142 is approximately equal to the distance between the electrodes as shown in FIG. 3b. The second piece of tubing 142 is followed by a second crimping ring 143 and a second piece of tubing 144. The two pieces of tubing 142 and 144 may be, for example, silicone rubber tubing. The size of the tubing 142 and 144 is not critical, but should be sized to be as small as possible while still having the capacity to hold the cables 140. The reason for being as small as possible is to minimize the diameter of the TCC probe 100 (as shown in FIG. 3c) to as small as possible. FIGS. 3b-c shows that the wire lead 115 adds to the diameter of the scope portion 105 of the TCC probe 100.

The area of the cables 140 over which the two crimping rings 141 and 143 are placed should be stripped of insulation so that electrical contact may be made between the crimping rings 141 and 143 and the cables 140. The crimping rings 141 and 143 may then be crimped to make this electrical contact. FIG. 5b shows the two crimping rings 141 and 143 crimped flat over the cables 140. The tubing 142 and 144 electrically isolates the crimping rings 141 and 143. The excess cabling 140 that is sticking out of crimping ring 141 may be trimmed. The electrodes (not shown) may then be welded to the crimping rings 141 and 143. As shown the crimping rings 141 and 143 are now flat and maybe welded to the inside of the electrodes, e.g., electrode 130 of FIG. 4. The electrodes may then be snapped onto the endoscopic portion 305 as described above. A clip for inserting the wire lead 115 into the power source may be placed onto the end of the cables 140 which extend from the second piece of tubing 144.

The fully constructed TCC probe 100 as shown in FIG. 3c may now be used on a patient to perform the transesophageal echocardiography procedure and the transesophageal cardioversion procedure. The patient may be administered an intravenous sedative and the TCC probe 100, starting with the echocardiography transducer 107 and the distal end 108, may be advanced through the patient's mouth into the esophagus to a depth consistent with the performance of the procedure (e.g., 35-40 cm from the tip of the echocardiography transducer 107 to the incisors). The transesophageal echocardiography procedure may then be performed in the known manner. In general, to perform the transesophageal echocardiography procedure, the transesophageal echocardiography portion of the TCC probe 100 (e.g., the echocardiography transducer 107) emits high frequency sound waves (ultrasound) and detects the echo of these sound waves. This data is transmitted to an external data processing device to produce an image of the structures of the heart as would be understood by those of skill in the art.

The doctor may then analyze the information obtained from the transesophageal echocardiography procedure to detennine whether a transesophageal cardioversion procedure should be performed. If the transesophageal cardioversion procedure is to be performed, the TCC probe 100 may then be repositioned (if necessary) to place the electrodes 112 and 113 in a selected location to provide a cardioversion current to the desired location of the heart. However, the TCC probe 100 may be designed such that no re-positioning is required. In order to perform the transesophageal cardioversion procedure, one or more additional electrodes are placed on the external chest cavity of the patient to provide a path for the electrical energy to travel along when it is emitted by the electrodes 112 and 113 of the TCC probe 100. Specifically, the electrical energy generated by the defibrillator travels along the wire lead 115 to the electrodes 112 and 113. As described above, the TCC probe 100 is placed within the esophagus at a position adjacent to the heart. The electrical energy is emitted from the electrodes 112 and 113 into the esophageal tissue and travels through to the heart. After passing through the heart, the electrical energy travels through the extracardiac tissue to the additional electrodes on the patient's chest. Since the esophagus is significantly closer to the heart than the exterior of the chest, less energy is required to be emitted from the TCC probe 100 than if a transthoracic cardioversion procedure was performed.

While the transesophageal cardioversion procedure is being performed, the transesophageal echocardiography portion of the TCC probe 100 may continue to be activated so that the doctor continues to receive images of the heart during and after the cardioversion procedure without inserting a second device into the patient. This allows the doctor to immediately see the results of the cardioversion procedure to evaluate the necessity of providing additional shocks to the heart and to determine if any complications have arisen as a result of the procedure. The patient experiences less discomfort because the same TCC probe 100 is used to perform both procedures.

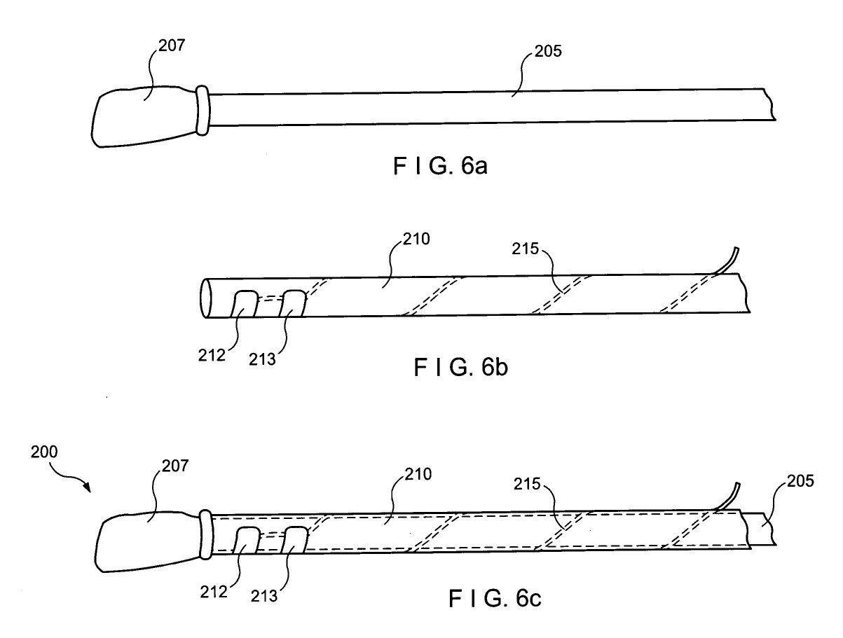

FIGS. 6a-c show various portions of an alternative exemplary embodiment of a TCC assembly 200. FIG. 6a shows a standard transesophageal echocardiography device which includes an echocardiography transducer 207 and a scope portion 205. FIG. 6b shows a specialized sheath 210 containing elements for a transesophageal cardioversion device according to the present invention. In operation, the sheath 210 is slipped over the scope portion 205 of FIG. 6a to position electrodes thereof for cardioversion. Those skilled in the art will understand that the sheath 210 according to this embodiment of the invention may be constructed of any flexible bio-compatible material, for example, a silicone tubing material.

In this embodiment, the sheath 210 includes two electrodes 212 and 213. These electrodes 212 and 213 serve the same purpose as electrodes 112 and 113 previously described and also share the same general dimensions and relative spacing as the previously described electrodes 112 and 113. The electrodes 212 and 213 may be embedded within the sheath 210 or fastened to the surface of the sheath 210. Of course, if desired, portions of the sheath 210 covering outer surfaces of the electrodes 212 and 213 may be removed to increase the efficiency of the transfer of the cardioversion current therefrom to the esophageal tissue. The electrodes 212 and 213 may, be constructed of any electrically conductive material. For example, the electrodes 212 and 213 may be formed from a thin titanium foil and may be fastened to the sheath 210 using medical adhesive. Of course those skilled in the art will understand that any of a wide range of conductors may be used for the electrodes 212 and 213 and that these electrodes may be fastened to the sheath 210 by any known method.

In addition, the sheath 210 also includes a wire lead 215 which serves the same function as described in regard to the wire lead 115 (i.e., carrying current from the defibrillator to the electrodes 212 and 213). The wire lead 215 generally runs on the interior of the sheath 210 so that the exterior of the sheath 210 presents a relatively smooth outer surface and represents the maximum outside diameter of the entire assembly. The conductors of the wire lead 215 are electrically connected to the electrodes 212 and 213 by, for example, soldering. At a far end of the sheath 210, the wire lead 215 separates from the sheath 210 so it may be connected to the defibrillator.

FIG. 6c shows a fully constructed TCC assembly 200 according to this exemplary embodiment. The TCC assembly is formed by sliding the sheath 210 over the scope portion 205 of the transesophageal echocardiography device. The sheath 210 is then coupled to the scope portion 205 to maintain the electrodes 212 and 213 in a desired position relative to the echocardiography probe 207. Those skilled in the art will understand that the sheath 210 may be coupled to the scope portion 205 in any number of ways. For example, the sheath 210 may be formed of an elastic material having an unstressed outer diameter which is less than an outer diameter of the scope portion 205. The sheath 210 is then stretched (e.g., by inflation) and drawn over the distal end of the scope portion 205 and aligned in a predetermined position. The sheath 210 is then released (e.g., by deflation) so that the sheath 210 shrinks around the scope portion 205 to hold it firmly in place thereon. Alternatively, one or both of the sheath 210 and the transesophageal echocardiography device may have a coupling mechanism mounted thereto (e.g., a clip or other locking mechanism) that holds the sheath 210 in the desired position after it has been slid over the scope portion 205.

Once the sheath 210 has been locked in place at the desired position on the scope portion 205, the TCC assembly 200 now has the capability of performing both transesophageal echocardiography and cardioversion procedures in the same manner as that described for the device of FIGS. 1-5. In addition, the TCC assembly 200 has the advantage that the sheath 210 may be disposed of after each procedure and a new sheath 210 provided for each subsequent procedure. Furthermore, this exemplary embodiment does not require purchase of a new TCC device nor does it require any modification to standard transesophageal echocardiography devices. The sheath 210 may be constructed with dimensions suited to any variety of existing transesophageal echocardiography devices so that a TCC assembly 200 may be formed with any transesophageal echocardiography device without any modification thereof.

In the preceding specification, the present invention has been described with reference to specific exemplary embodiments thereof. It will, however, be evident that various modifications and changes may be made thereunto without departing from the broadest spirit and scope of the present invention as set forth in the claims that follow. The specification and drawings are accordingly to be regarded in an illustrative rather than restrictive sense.

1. A medical apparatus comprising:
a flexible probe for accessing a patient's esophagus via the mouth, the probe, when in an operative position extending from a proximal end which remains outside the patient to a distal end within the esophagus;
an echocardiography transducer coupled to the distal end of the probe so that, when the probe is in the operative position, the echocardiography transducer is at a predetermined location within the esophagus relative to the heart to perform a transesophageal echocardiography procedure; and
an electrode disposed on the probe for delivering a cardioversion current to the heart via the esophagus.

2. The apparatus of claim 1, wherein the electrode comprises a plurality of electrodes disposed on the probe, each of the electrodes being coupled to a wire lead extending along the probe to the proximal end to couple to a power source.

3. The apparatus of claim 2, wherein the power source is one of a defibrillator and a cardioverter.

4. The apparatus of claim 1, wherein the apparatus is used to treat cardiac arrhythmia.

5. The apparatus of claim 2, wherein the electrodes are spaced along a longitudinal axis of the probe and wherein the electrodes are coupled to the power source via a plurality of leads so that the selected ones of the are energized to supply cardioversion current to portions of the heart located adjacent to the selected ones of the electrodes.

6. The apparatus of claim 1, wherein the electrode is selectively mountable on and removable from the scope portion.

7. The apparatus of claim 1, wherein the electrode is mounted to a flexible sheath which is sized to be received over a distal portion of the probe and fixed thereon at a predetermined location, and wherein, when the sheath is fixed at the predetermined location, the electrode is in a desired position relative to the echocardiography transducer.

8. The apparatus of claim 7, wherein the sheath is permanently bonded to the probe.

9. The apparatus of claim 7, wherein the sheath is selectively mountable on and removable from the probe.

10. The apparatus of claim 9, wherein the electrode is coupled to a wire lead which extends from the electrode along the probe to exit the patient's body and couple to a power source.

11. The apparatus of claim 1, wherein the electrode is formed of a titanium foil.

12. The apparatus of claim 1, wherein the electrode has a length of 7-10 mm along an axial direction of the probe.

13. The apparatus of claim 2, wherein a proximal end of a first electrode is separated from a proximal end of a second electrode by a distance of 5-8 mm.

14. A cardioversion mechanism comprising an electrode assembly selectively mountable to a transesophageal echocardiography probe, wherein, when mounted to the echocardiography probe, electrodes of the electrode assembly are fixed at a predetermined location with respect to the echocardiography probe, the electrode assembly being coupled to a power source for supplying a cardioversion current to heart via tissue located adjacent thereto when the echocardiography probe is in an operative position within an esophagus of a patient.

15. The cardioversion mechanism of claim 14, wherein the electrode assembly is one of a single use assembly and a multiple use assembly.

16. The cardioversion mechanism of claim 14, wherein the electrode assembly includes a sheath for mounting the electrode assembly to the echocardiography probe.

17. The cardioversion mechanism of claim 16, wherein the electrodes and at least one lead wire coupling the electrodes to the power source are mounted one of within the sheath and on the sheath.

18. The cardioversion mechanism of claim 16, wherein the sheath is a flexible condom material for mounting to the echocardiography probe.

19. The cardioversion mechanism of claim 14, wherein the echocardiography probe includes a flexible insertion portion and an echocardiography transducer portion coupled to the flexible insertion portion.

20. A method of treating a heart of a patient, comprising the steps of:
inserting into the patient's esophagus a device comprising a flexible probe having an echocardiography transducer coupled to a distal end thereof and at least one cardioversion electrode coupled to the probe;
performing an echocardiography to analyze a condition of the heart; and
applying electric current to the at least one electrode to supply a cardioversion current to the heart when the echocardiography does not contraindicate cardioversion.

21. The method of claim 20, further comprising the step of performing an additional echocardiography immediately after the cardioversion using the echocardiography transducer.

22. The method of claim 20, further comprising the step of, prior to inserting the device into the esophagus, removably coupling a sheath to a distal portion of the probe, wherein the at least one electrode is mounted to the sheath.

23. The method of claim 22, further comprising the step of disposing the sheath after completing the procedure.
